(19) United States
(12) Patent Application Publication
(10) Pub. No.: US 2016/0113622 A1
(43) Pub. Date: Apr. 28, 2016

(54) CATHETER FOR DETECTION OF ULTRASOUND AND PHOTOACOUSTIC SIGNALS AND ULTRASOUND/PHOTOACOUSTIC IMAGE ACQUISITION SYSTEM USING THE SAME

(71) Applicant: POSTECH ACADEMY-INDUSTRY FOUNDATION, Pohang-si (KR)

(72) Inventors: Chul Hong KIM, Pohang-si (KR); Sung Jo PARK, Daegu (KR); Chang Hoon CHOI, Yongin-si (KR)

(21) Appl. No.: 14/878,271

(22) Filed: Oct. 8, 2015

(30) Foreign Application Priority Data
Oct. 23, 2014 (KR) 10-2014-0144082

(51) Int. Cl. A61B 8/00; A61B 5/00; A61B 8/08; A61B 8/12

(52) U.S. Cl. CPC A61B 8/4416; A61B 8/12; A61B 5/0095; A61B 8/5261; A61B 8/445; A61B 8/4483

(57) ABSTRACT

Provided are a catheter for detection of ultrasound and photoacoustic signals and an image acquisition system using the catheter. The catheter for detection of ultrasound and photoacoustic signals includes a lens optical fiber where a lens is attached to an end of an optical fiber, an ultrasound transducer, a catheter main body where the lens optical fiber and the cable are disposed to pass through an inner portion thereof, a catheter head which is connected to an end of the catheter main body, and a membrane which surrounds a surface of the catheter head.

The catheter is configured so as to irradiate condensed light by using the lens optical fiber and to receive the photoacoustic signal or to receive and transmit the ultrasound signal. Accordingly, it is possible to simultaneously acquire an ultrasound image and a high-resolution photoacoustic image.

### CROSS-REFERENCE TO RELATED PATENT APPLICATION

This application claims the benefit of Korean Patent Application No. 10-2014-0144082, filed on Oct. 23, 2014, in the Korean Intellectual Property Office, the disclosure of which is incorporated herein in its entirety by reference.

### BACKGROUND OF THE INVENTION

1. Field of the Invention

The present invention relates to a catheter for detection of ultrasound and photoacoustic signals and, more particularly, to an endoscopic catheter capable of simultaneously an ultrasound signal and a photoacoustic signal and an image acquisition system capable of acquiring a superimposed image of ultrasound and photoacoustic images by using the endoscopic catheter.

2. Description of the Prior Art

In the related art, a blood vessel disease diagnosis apparatus using ultrasound inside a blood vessel displays a current position and an ultrasound image of a cross section of an inner wall of the blood vessel by using the ultrasound. By using the ultrasound image of the cross section of the inner wall of the blood vessel, a degree of angiostenosis of the blood vessel and a type, length, and state of lesion can be stereoscopically identified in quantitative and qualitative manners. However, in this method of the related art, a structure and plaque of the inner wall of the blood vessel are merely displayed through the ultrasound image. Therefore, since physiological information of inner tissue of the blood vessel cannot be identified, there is a problem in that it is difficult to accurately determine physiological components of the lesion. In particular, there is a problem in that a vulnerable plaque causing acute myocardial infarction cannot be accurately identified by using only the ultrasound image.

On the other hand, a photoacoustic effect occurs as follows. A cellular tissue is allowed to absorb optical energy. The cellular tissue converts the light energy to heat energy. The cellular tissue is thermally expanded by the converted heat energy, and the photoacoustic signal is generated by the thermal expansion. The photoacoustic signal is detected, and a 3-D image of the cellular tissue is generated. Information which can be obtained from the photoacoustic effect includes lipid, melanin, hemoglobin oxygen saturation, total hemoglobin concentration, or other physiological information of the cellular tissue.

However, in the related art, there is a problem in that an apparatus for acquiring the photoacoustic image cannot be easily manufactured as an endoscopic catheter because mirrors, lenses, light sources, and other optical members has complicated structures and large sizes. In addition, in the related art, in the case of using the apparatus for acquiring the photoacoustic image, peripheral devices for obtaining a high-resolution image are complicated, so that it is difficult to manufacture a miniaturized apparatus.

### SUMMARY OF THE INVENTION

The present invention is to provide an endoscopic catheter capable of simultaneously detecting an ultrasound signal and a photoacoustic signal.

The present invention is also to provide an ultrasound/photoacoustic image acquisition system capable of simultaneously acquiring an ultrasound image and a photoacoustic image by using the endoscopic catheter.

According to a first aspect of the present invention, there is provided a catheter for detection of ultrasound and photoacoustic signals including a lens optical fiber where a lens is attached to an end of an optical fiber, an ultrasound transducer which receives and transmits the ultrasound signal, a cable which is connected to the ultrasound transducer to receive and transmit signals, a catheter main body where the lens optical fiber and the cable are disposed to pass through an inner portion thereof, a catheter head which is connected to an end of the catheter main body, and a membrane which surrounds a surface of the catheter head, wherein the end of the lens optical fiber where the lens is attached and the ultrasound transducer are fixedly attached to the catheter head, and the ultrasound transducer receives the ultrasound signal and the photoacoustic signal.

In an ultrasound/photoacoustic image acquisition system according to the present invention, by combining ultrasound diagnosis and a photoacoustic effect, it is possible to obtain various advantages and functional effects of an ultrasound diagnosis method for an inner portion of a blood vessel of the related art and to obtain various advantages of acquisition of physiological information, good contrast between light and dark, spatial resolution, and the like due to the photoacoustic effect. As a result, in comparison with methods or apparatus of the related art, it is possible to more accurately diagnose disease in the inner portion of the blood vessel based on the ultrasound image and the photoacoustic image.

In addition, in the ultrasound/photoacoustic image acquisition system according to the present invention, the variable wavelength pulse laser, the lens optical fiber, the miniaturized ultrasound transducer, and the endoscopic catheter are used without addition of complicated peripheral devices, so that the ultrasound/photoacoustic image acquisition system can be easily manufactured. In addition, the lens optical fiber integrated with the lens is used, so that it is possible to overcome a complication of a structure and a limitation in size caused by internal optical members of an existing endoscope, to relatively reduce the size of the catheter, and to obtain a high-resolution image.

In addition, due to a difference in structure from the catheter of the related art, the size of the endoscopic catheter according to the present invention is remarkably reduced. In particularly, in the catheter according to the present invention, the lens optical fiber is used for transmitting laser, and the lens optical fiber focuses the emitted light in a predetermined direction by the lens. In this manner, if the lens optical fiber is used, the lens of the optical fiber end adjusts the direction and focus of the light, and thus, there is no need for an additional mirror. In addition, without an additional lens for condensing the light, a high-resolution photoacoustic image can be obtained.

Due to these features of the lens optical fiber, it is possible to solve the problem of the related art in that the size of the catheter is increased by the optical members, and it is possible to obtain a high-resolution image.

In the catheter according to the present invention, a diameter thereof can be reduced down to 1 mm or less, and various applications are available by using customized miniaturized ultrasound transducer and lens optical fiber according to use purposes and methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram illustrating overall configuration of ultrasound/photoacoustic image acquisition system using a catheter for detection of an ultrasound signal and a photoacoustic signal according to the present invention.

FIG. 2 is a perspective diagram illustrating a catheter head in a catheter for detection of ultrasound and photoacoustic signals according to the present invention.

FIGS. 3A, 3B, and 3C are a perspective diagram and vertical and horizontal cross-sectional diagrams illustrating the catheter for detection of the ultrasound signal and the photoacoustic signal according to the present invention.

FIG. 4 is a cross-sectional diagram illustrating a lens optical fiber in a catheter according to the present invention.

FIG. 5 is a diagram illustrating reception of a photoacoustic signal in the catheter according to the present invention.

FIG. 6 is a graph illustrating received ultrasound and photoacoustic signals in a time-voltage relation in an ultrasound/photoacoustic image acquisition system according to the present invention.

FIG. 7 is a cross-sectional diagram illustrating a state of experiment configured to acquire an ultrasound image and a photoacoustic image by using the ultrasound/photoacoustic image acquisition system according to the present invention, wherein a catheter head is inserted into a phantom that is an experiment model.

FIG. 8 is a diagram illustrating the photoacoustic image (PA Image) and the ultrasound image (US Image) acquired by rotating the catheter head by 360° in the state of FIG. 7.

FIG. 9 is a diagram illustrating an image obtained by superimposing the photoacoustic and ultrasound images of FIG. 8.

FIG. 10 is a cross-sectional diagram illustrating a state of experiment configured to acquire an ultrasound image and a photoacoustic image by using the ultrasound/photoacoustic image acquisition system according to the present invention, wherein a catheter head is inserted into a phantom that is an experiment model.

FIG. 11 is a diagram illustrating the photoacoustic image (PA Image) and the ultrasound image (US Image) acquired by rotating the catheter head over the range of 360 degrees in the state of FIG. 10.

FIG. 12 is a diagram illustrating an image obtained by superimposing the photoacoustic and ultrasound images of FIG. 11.

FIG. 13 is a cross-sectional diagram illustrating a state of experiment configured to acquire an ultrasound image and a photoacoustic image by using the ultrasound/photoacoustic image acquisition system according to the present invention, wherein a catheter head is inserted into a phantom that is an experiment model.

FIG. 14 is a diagram illustrating the photoacoustic image (PA Image) and the ultrasound image (US Image) acquired by rotating the catheter head over the range of 360 degrees in the state of FIG. 13.

FIG. 15 is a diagram illustrating an image obtained by superimposing the photoacoustic and ultrasound images of FIG. 14.

### DETAILED DESCRIPTION OF THE INVENTION

A catheter according to the present invention and an ultrasound/photoacoustic image acquisition system can simplify a structure, minimize a size, and improve resolution by using a lens optical fiber and can more accurately diagnose lesion in a blood vessel by simultaneously acquiring an ultrasound image and a photoacoustic image.

Hereinafter, a configuration and operations of the ultrasound/photoacoustic image acquisition system using a catheter for detection of ultrasound and photoacoustic signals according to the present invention will be described in detail.

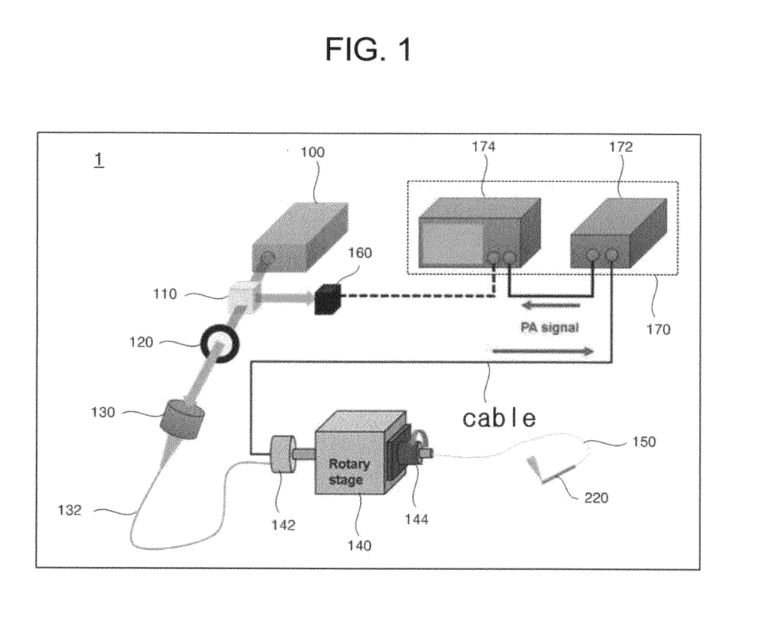

FIG. 1 is a diagram illustrating overall configuration of the ultrasound/photoacoustic image acquisition system using the catheter for detection of ultrasound and photoacoustic signals according to the present invention. Referring to FIG. 1, the ultrasound/photoacoustic image acquisition system 1 according to the present invention is configured to include a light source 100, a beam splitter 110, a lens 120, a collimator 130, an optical fiber 132, a rotation stage 140, a catheter 150, a photodetector 160, and a signal output unit 170.

The light source 100 provides light for acquiring the photoacoustic signal. A variable wavelength pulse laser (tunable pulse laser) may be used as the light source. Preferably, the image acquisition system according to the present invention compares light absorbance of to be-diagnosed cell tissue and lesion according to wavelength based on a fact that the light absorbance is different among materials, and the image acquisition system selects an appropriate laser wavelength to obtain the photoacoustic signal of a target.

The beam splitter 110 splits the light output from the light source into first light and second light. The first light is allowed to pass through the lens 120 for maintaining a beam width and the collimator 130 for collimation. After that, the first light is provided to the optical fiber 132. The second light is detected by the photodetector 160 to be converted to an electrical signal to be transmitted to the signal output unit 170 so that the second light is used as a reference signal for analysis of the photoacoustic signal.

The catheter 150 receives and transmits the ultrasound signal or receives the photoacoustic signal to transmit the signals to the signal output unit. The catheter 150 is configured so that the end of the lens optical fiber and the ultrasound transducer are fixedly attached to the catheter head. The catheter irradiates light through the lens optical fiber and receives the ultrasound signal or the photoacoustic signal through the ultrasound transducer. The catheter transmits the received signals to the signal output unit. The structure of the catheter will be described later.

The light of the light source is transmitted through the optical fiber 132 to the lens optical fiber of the catheter and emits through the lens of the lens optical fiber in the direction directed by the ultrasound transducer. The photodetector 160 detects second light provided from a beam splitter. Next, the photodetector converts the light to an electrical signal to transmit the signal to the signal output unit.

The signal output unit 170 is connected to the catheter and the photodetector. The signal output unit receives a reference signal from the photodetector and receives the ultrasound signal and/or the photoacoustic signal from the ultrasound transducer of the catheter to convert the signal to voltage and outputs the voltage.

The signal output unit 170 is configured to include a pulser receiver 172 and an oscilloscope 174. The pulser receiver receives the photoacoustic signal (PA signal) and the ultrasound signal (US signal) and amplifies the photoacoustic signal and the ultrasound signal to transmit the signals to the oscilloscope. The oscilloscope converts the receive photoacoustic and ultrasound signals to voltage values and processes these values to generate an ultrasound image and a photoacoustic image. The oscilloscope transmits the ultrasound image and the photoacoustic image to a screen or the like.

On the other hand, preferably, a rotation stage 140 where a rotary joint 142 and a jig 140 which can be rotated over the range of 360 degrees are attached to two ends thereof is included. An end of the catheter main body is connected to the jig, and the optical fiber and the cable of the signal output unit are connected to the rotary joint, so that the lens optical fiber and cable connected to the ultrasound transducer is prevented from being damaged by a rotational force applied during the rotation even though the catheter is rotated over the range of 360 degrees.

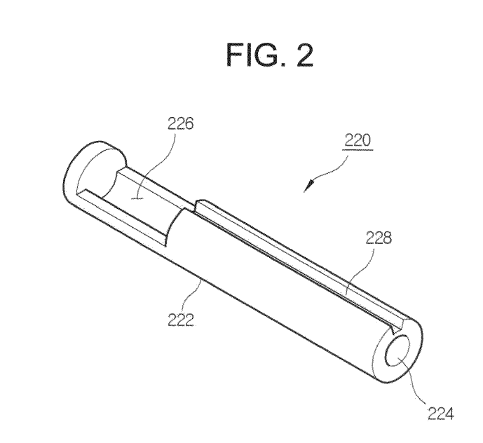

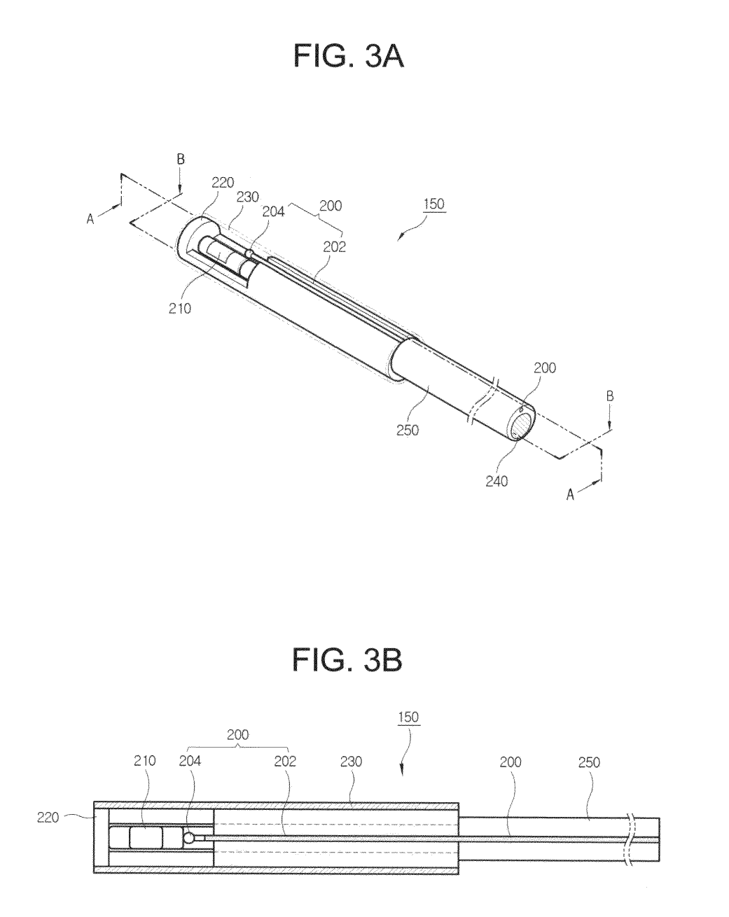

Hereinafter, a structure and operations of the catheter 150 for detection of ultrasound and photoacoustic signals according to the present invention will be described more in detail with reference to FIG. 2 and FIGS. 3A, 3B, and 3C.

FIG. 2 is a perspective diagram illustrating the catheter head in the catheter for detection of ultrasound and photoacoustic signals according to the present invention. FIGS. 3A, 3B, and 3C are a perspective diagram and vertical and horizontal cross-sectional diagrams illustrating the catheter for detection of the ultrasound signal and the photoacoustic signal according to the present invention.

Referring to FIGS. 2 to FIGS. 3A, 3B, and 3C, the catheter 150 for detection of ultrasound and photoacoustic signals according to the present invention is configured to include a lens optical fiber 200, an ultrasound transducer 210, a catheter head 220, a membrane 230, a cable 240 connected to the ultrasound transducer, and a catheter main body 250.

The lens optical fiber 200 is configured so that a lens 204 is attached to an end of the optical fiber 202. The ultrasound transducer 210 receives and transmits the ultrasound signal or receives the photoacoustic signal. The ultrasound transducer transmits the received ultrasound and photoacoustic signals to the signal output unit through the connected cable 240.

The catheter main body 250 is formed so that the inner portion becomes a hollow tube, and the cable connected to the ultrasound transducer and the lens optical fiber are disposed in the inner portion of the catheter main body.

The catheter head 220 is connected to an end of the catheter main body, and the ultrasound transducer is fixedly attached to the end of the lens optical fiber. The catheter head is configured with a metal which is enough rigid to perform precision processing, and for example, brass, stainless steel, special aluminum, or the like may be used.

The membrane 230 is configured to surround the surface of the catheter head. Preferably, in order to reduce attenuation of the ultrasound signal during the transmission inside the catheter head, a space between the head and the membrane is filled with deionized water. The membrane is configured with a chemical ultrathin film surrounding the catheter head in order to protect the lens optical fiber of the catheter head and the ultrasound transducer. In addition, preferably, the membrane is configured so that a wall of a blood vessel is not damaged in the inner portion of the human body.

The chemical ultrathin film is a chemical material of a very thin film constituting the aforementioned membrane. As an example, LDPE (low-density polyethylene) which is solid at the room temperature may be used. Preferably, the membrane is an ultra-thin film having a thickness of about 10 to 20 micrometers and low density so that the membrane is very flexible. In addition, preferably, the membrane has a low degree of attenuation of the ultrasound signal, so that the ultrasound signal can be transmitted, a transparent property is good, and light can be transmitted. Preferably, the chemical ultrathin film constituting the membrane surrounds the catheter, so that damage to the catheter caused by external collision is prevented and, at the same time, damage to a wall of a blood vessel in the inner portion of the human body is prevented. In addition, the deionized water filling the space between the catheter head and the membrane is surrounded by the chemical ultrathin film constituting the membrane, so that the deionized water does not leak out.

Referring to FIG. 2, the catheter head 220 is configured to include a head main body 222 having a predetermined length, a hollow portion 224 which is formed in an inner portion thereof along a longitudinal direction of the head main body, an opening portion 226 which is formed to be connected to an end of the hollow portion, and a groove 228 which is formed on a surface thereof along the longitudinal direction of the head main body.

Preferably, the ultrasound transducer is attached to an end of the hollow portion 224 and is disposed to be directed to the opening portion 226 so as not to obstruct acquisition of the ultrasound signal. Preferably, the cable connected to the ultrasound transducer is disposed to pass through the hollow portion.

Preferably, the lens optical fiber 200 are attached to the groove, the ultrasound transducer and the lens of the lens optical fiber are disposed so that front surfaces thereof are directed to the opening portion 226 of the catheter head. In particular, preferably, the lens of the lens optical fiber is disposed so that light is irradiated in the direction where the ultrasound transducer is directed. Preferably, the groove of the catheter head is formed in a shape of a V groove so that the lens optical fiber can be fixed.

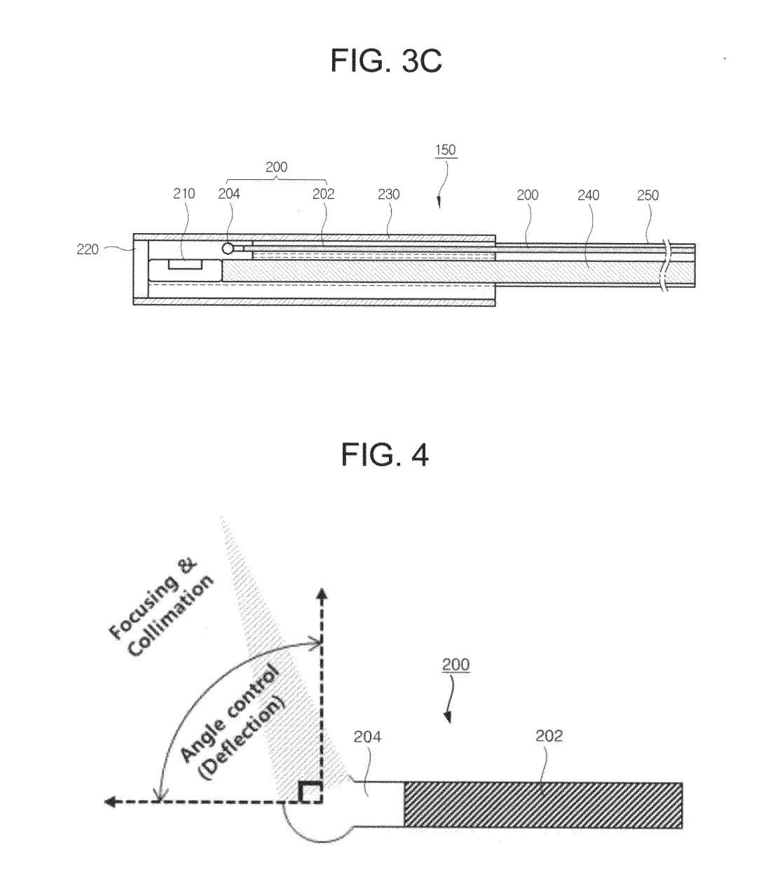

FIG. 4 is a cross-sectional diagram illustrating the lens optical fiber in the catheter according to the present invention. Referring to FIG. 4, the lens optical fiber is configured by attaching a lens to the end of the optical fiber, and a focal length and an emitting angle of the lens can be adjusted according to desired conditions at the time of manufacturing. Therefore, preferably, the focal length and the emitting angle of the lens are adjusted according to a distance separated from a target which is to be diagnosed or measured.

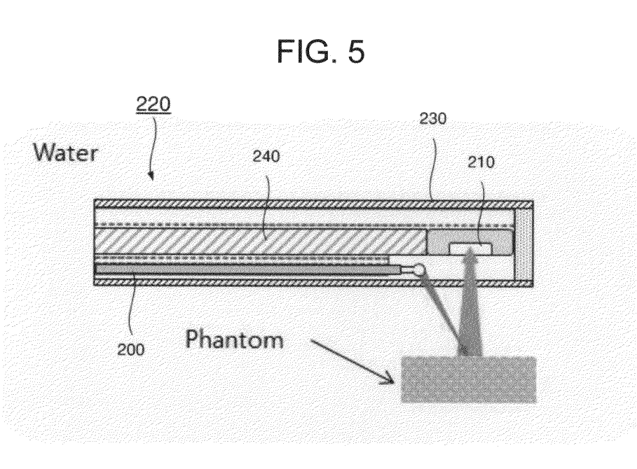

FIG. 5 is a diagram illustrating reception of a photoacoustic signal in the catheter according to the present invention. Referring to FIG. 5, variable wavelength pulse laser is projected to a phantom in water through the lens optical fiber to supply light energy to the phantom, and the photoacoustic signal emitted from the phantom is received by using the ultrasound transducer. In FIG. 5, the separation distance between the phantom and the ultrasound transducer is 3 mm.

Hereinafter, operations of the ultrasound/photoacoustic image acquisition system having the above-described configuration according to the present invention will be described in brief.

First, the variable wavelength pulse laser as alight source supplies light of a specific wavelength. The light is split by the beam splitter. Some of the light is transmitted to the photodetector used for receiving a signal trigger, and the other of the light is incident on the optical fiber through a lens and a collimator. The laser is emitted from the end of the catheter head through the optical fiber and the lens optical fiber. The photoacoustic signal which is generated through light absorption of a target is acquired by the ultrasound transducer of the catheter head. The photoacoustic signal is transmitted through the cable to the pulser receiver to be amplified. The photoacoustic signal is converted to a voltage value by the oscilloscope, and the photoacoustic signal is image-processed to be displayed as a photoacoustic image. In this case, the catheter can be rotated over the range of 360 degree by the jig connected to the rotation stage if required, and at the time, the lens optical fiber and the cable are prevented from being damaged by a rotational force applied at this time by using the rotary joint.

On the other hand, in the case where the inner portion of the blood vessel is to be diagnosed by using the image acquisition system according to the present invention, the lens optical fiber transmits light, the end of the lens optical fiber emits the light to focus the light at a desired point of the inner portion of the blood vessel to condensely irradiate the point with the light. The emitted light is absorbed by the wall of the blood vessel to be converted to heat energy. The heat energy leads to thermal expansion, and thus, the photoacoustic signal is generated by the thermal expansion. The ultrasound transducer acquires the generated photoacoustic signal to convert a voltage value and transmits the voltage value to a data processing system, so that the photoacoustic image can be obtained. In addition, the ultrasound image can be obtained together with the photoacoustic image. Therefore, it is possible to obtain physiological information on the tissue inside the blood vessel which cannot easily obtained merely by using the ultrasound inside the blood vessel. Preferably, the catheter is configured so that the outer portion is surrounded by a chemical fiber of a thin special material to protect the catheter and the inner portion of the blood vessel of the human body.

According to the present invention, it is possible to obtain various advantages and functional effects of the ultrasound diagnosis method of the related art and to more accurately diagnose disease of the blood vessel by combining various advantages of acquisition of physiological information, good contrast between light and dark, spatial resolution, and the like due to the photoacoustic effect. In addition, according to the present invention, the variable wavelength pulse laser, the lens optical fiber, the miniaturized ultrasound transducer, and the endoscopic catheter are used without addition of complicated peripheral devices, so that the ultrasound/photoacoustic image acquisition system can be easily manufactured. In addition, the lens optical fiber is used, so that it is possible to overcome a complication of a structure and a limitation in size caused by internal optical members of an existing endoscope, to relatively reduce the size of the catheter, and to obtain a high-resolution image.

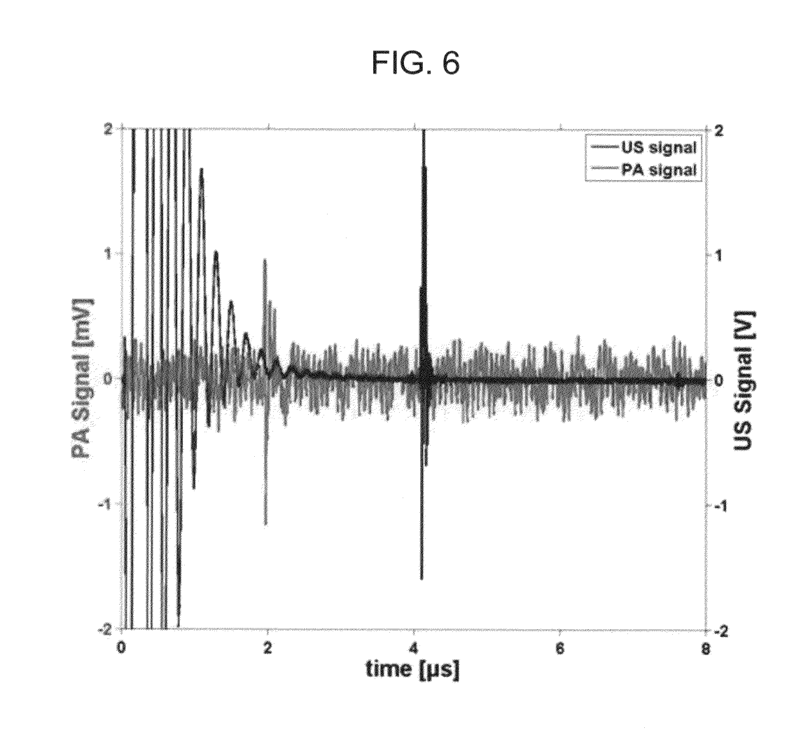

FIG. 6 is a graph illustrating the received ultrasound and photoacoustic signals in a time-voltage relation in the ultrasound/photoacoustic image acquisition system according to the present invention. Referring to FIG. 6, in the image acquisition system according to the present invention, like FIG. 5, light is emitted to the phantom through the lens optical fiber, and thus, the photoacoustic signal (PA Signal) is received by the ultrasound transducer. In addition, the ultrasound signal is transmitted to the phantom through the ultrasound transducer, and thus, the ultrasound signal (US Signal) is received. The two experiments are performed in water, and the speed of ultrasound is theoretically 1500 m/sec in water. The time of about 2 μs is taken for the ultrasound to propagate 3 mm. In this experiment, since the round-trip distance is 6 mm, the time of about 4 μs is taken, which can be identified from the black ultrasound graph. In the case of the experiment of acquiring the photoacoustic signal, since the speed of the laser is theoretically the speed of light 3×10⁸ m/sec, the speed of the laser is much larger than the speed of ultrasound. As a result, the time taken for the laser to reach the phantom is negligibly shorter than the time taken for the photoacoustic signal which is ultrasound to reach the ultrasound transducer. The time taken from the generation of the laser pulse to the acquisition of the signal is 2 μs which is the time taken for only the photoacoustic signal to reach the ultrasound transducer. This can be identified from the red photoacoustic signal graph.

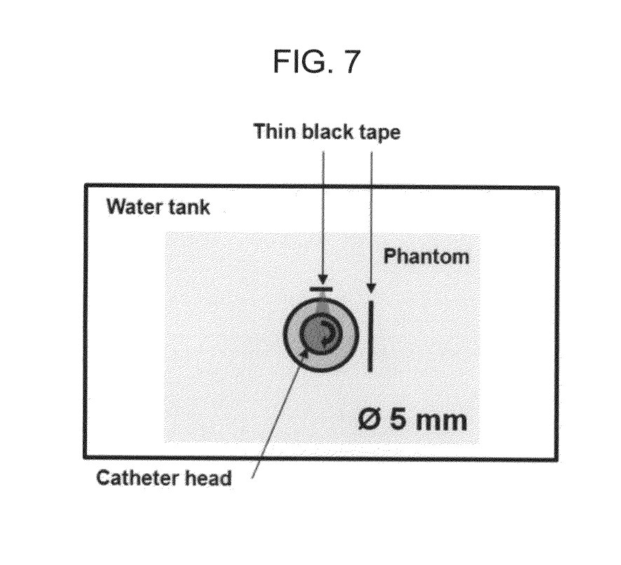

On the other hand, referring to FIGS. 7 to 13, it will be described that a type of the lesion can be identified by checking physiological information on the blood vessel and the shape of the blood vessel based on the images acquired by using the ultrasound/photoacoustic image acquisition system according to the present invention.

FIG. 7 is a cross-sectional diagram illustrating a state of experiment configured to acquire an ultrasound image and a photoacoustic image by using the ultrasound/photoacoustic image acquisition system according to the present invention, wherein the catheter head is inserted into the phantom that is an experiment model. In the configuration of the experiment illustrated in the cross-sectional diagram of FIG. 7, a phantom simulating a human cell is disposed in a water tank, the catheter head is located inside a hole which is punctured in a tubular shape having a diameter of 5 mm in the phantom to measure an image. In particular, a thin black tape in a shape of a thin film is fixed inside the phantom.

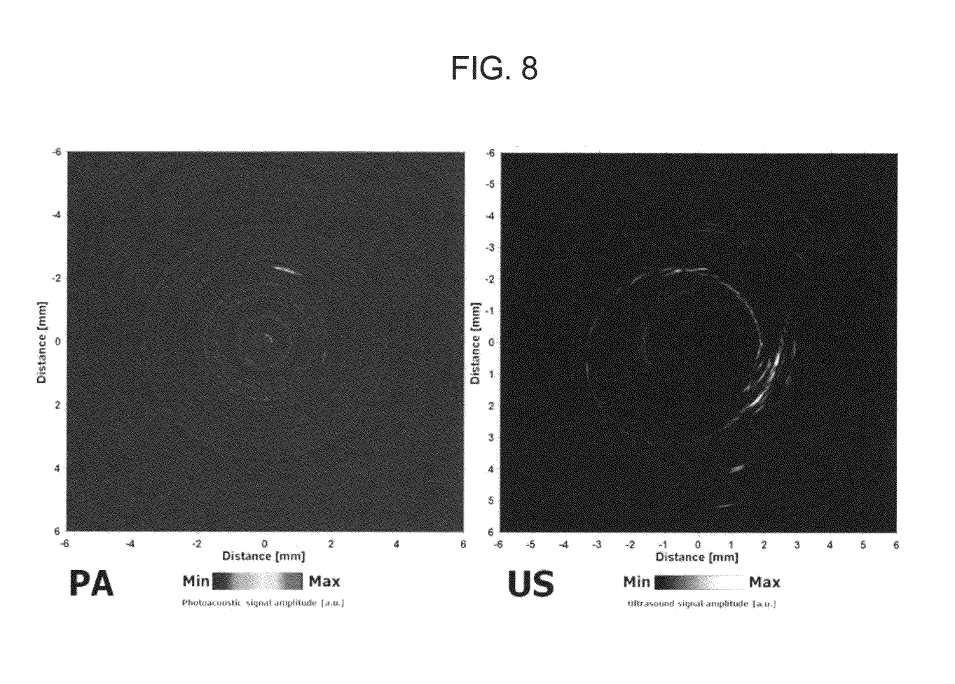

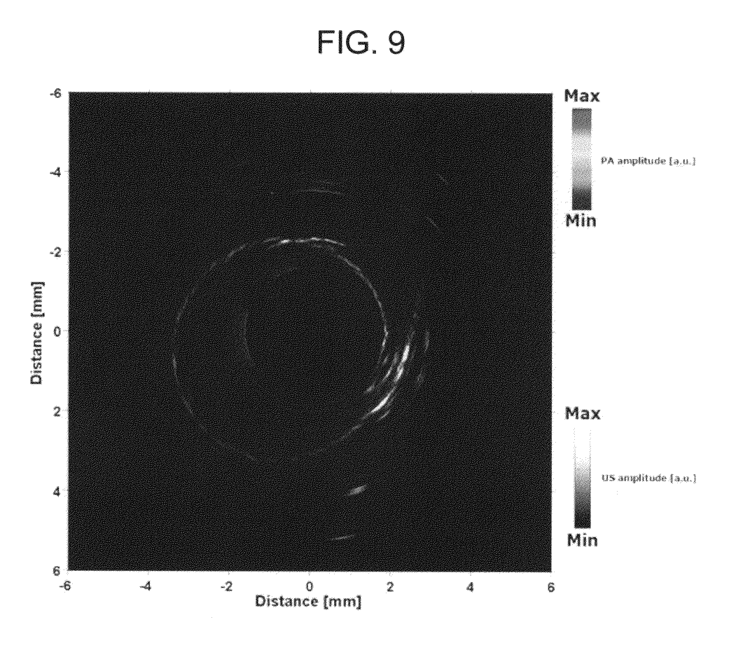

FIG. 8 is a diagram illustrating the photoacoustic image (PA Image) and the ultrasound image (US Image) acquired by rotating the catheter head over the range of 360 degrees in the state of FIG. 7. FIG. 9 is a diagram illustrating an image obtained by superimposing the photoacoustic and ultrasound images of FIG. 8. It can be seen from the photoacoustic image of FIG. 8 that the phantom does not react and the black tape inside the phantom receives the laser, so that the photoacoustic signal is extracted to identify the tape. On the other hand, it can be seen from the ultrasound image of FIG. 8 that the shape of the hole in the phantom is detected but the black tape is not detected. It can be seen from the image of FIG. 9 obtained by superimposing the photoacoustic image and the ultrasound image of FIG. 8 that both of the shape of the hole in the phantom and the black tape are detected.

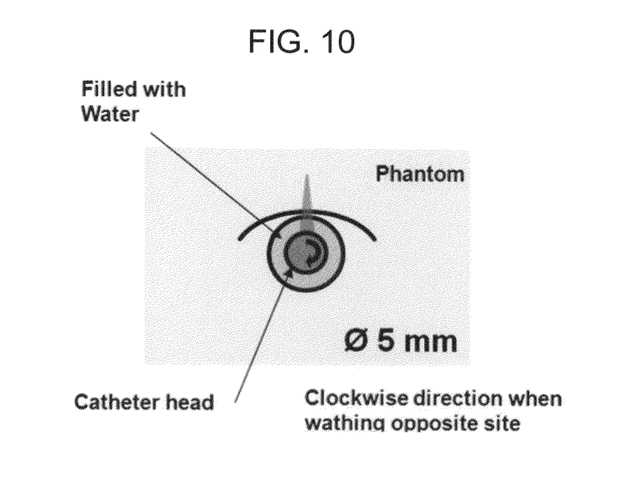

In addition, FIG. 10 is a cross-sectional diagram illustrating a state of experiment configured to acquire an ultrasound image and a photoacoustic image by using the ultrasound/photoacoustic image acquisition system according to the present invention, wherein the catheter head is inserted into the phantom that is an experiment model. It can be seen from the experiment state of FIG. 10 that the black tape is located to be elongated inside the phantom.

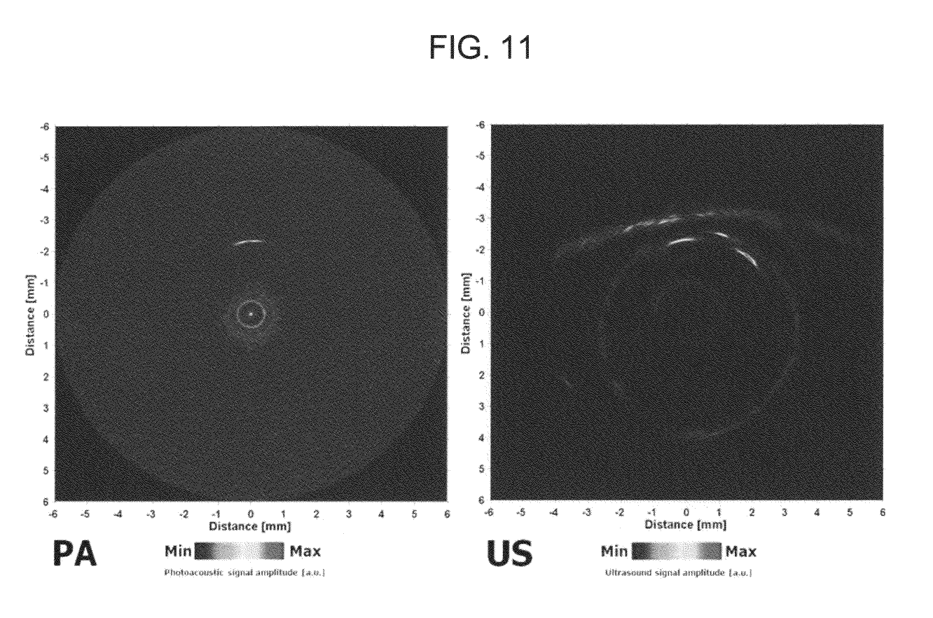

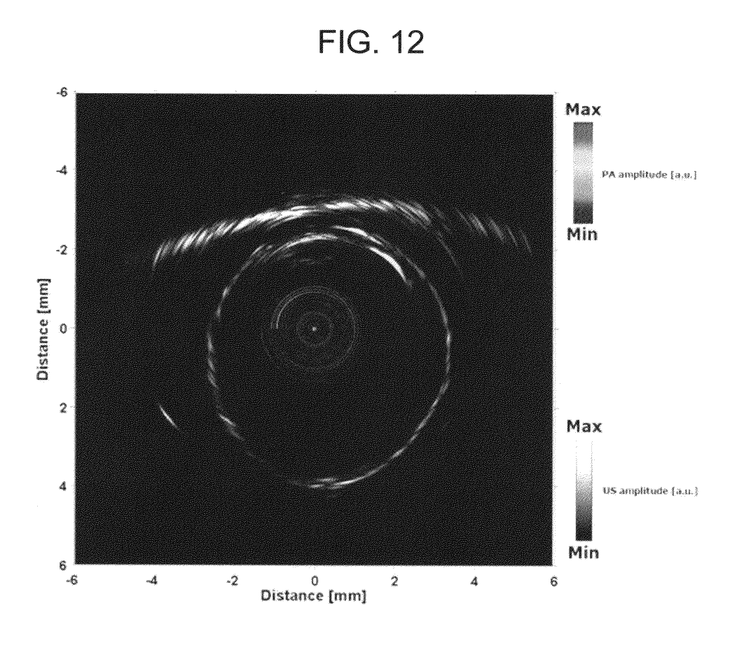

FIG. 11 is a diagram illustrating the photoacoustic image (PA Image) and the ultrasound image (US Image) acquired by rotating the catheter head over the range of 360 degrees in the state of FIG. 10. FIG. 12 is a diagram illustrating an image obtained by superimposing the photoacoustic and ultrasound images of FIG. 11. In FIGS. 11 to 12, a shape of a hole and a black tape of the phantom can be identified from the image obtained by superimposing the photoacoustic image and the ultrasound image.

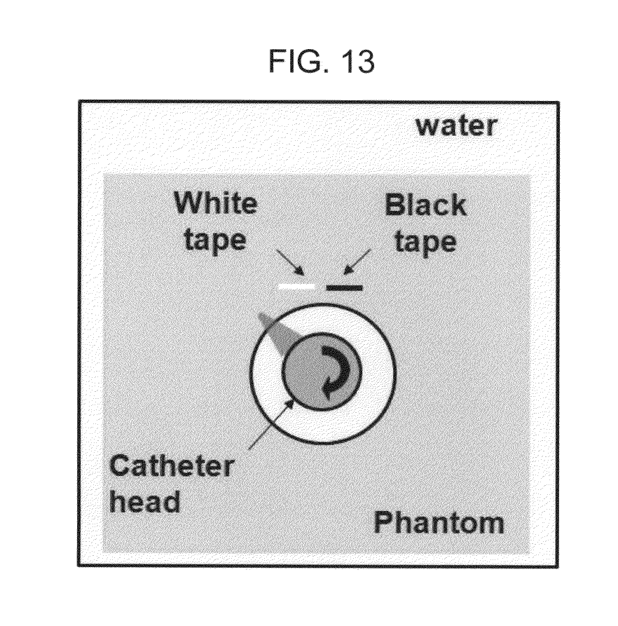

In addition, FIG. 13 is a cross-sectional diagram illustrating a state of experiment configured to acquire an ultrasound image and a photoacoustic image by using the ultrasound/photoacoustic image acquisition system according to the present invention, wherein the catheter head is inserted into the phantom that is an experiment model. It can be seen from the experiment state of FIG. 13 that a black tape and a white tape are located in front of the catheter head inside the phantom.

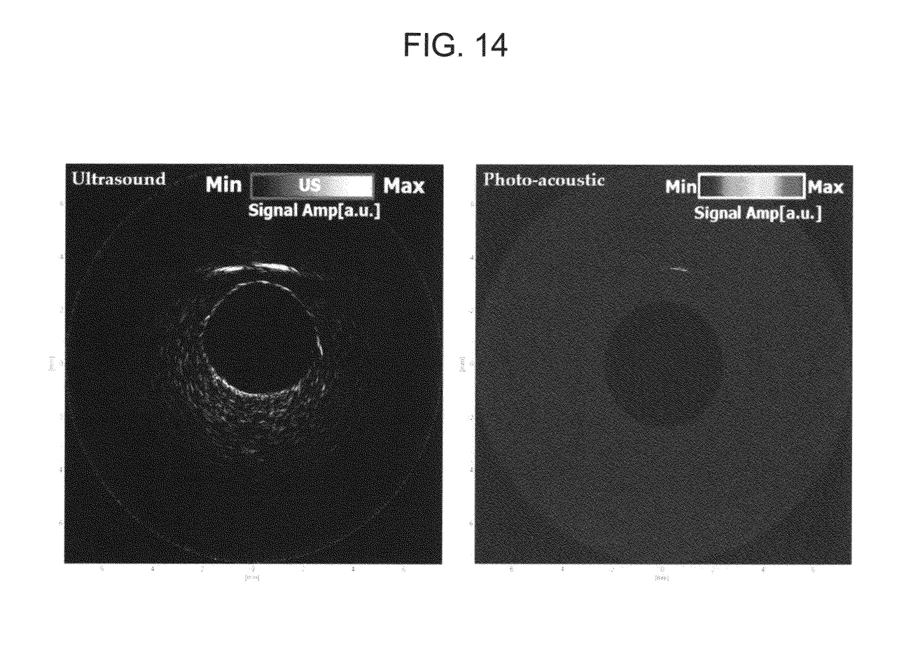

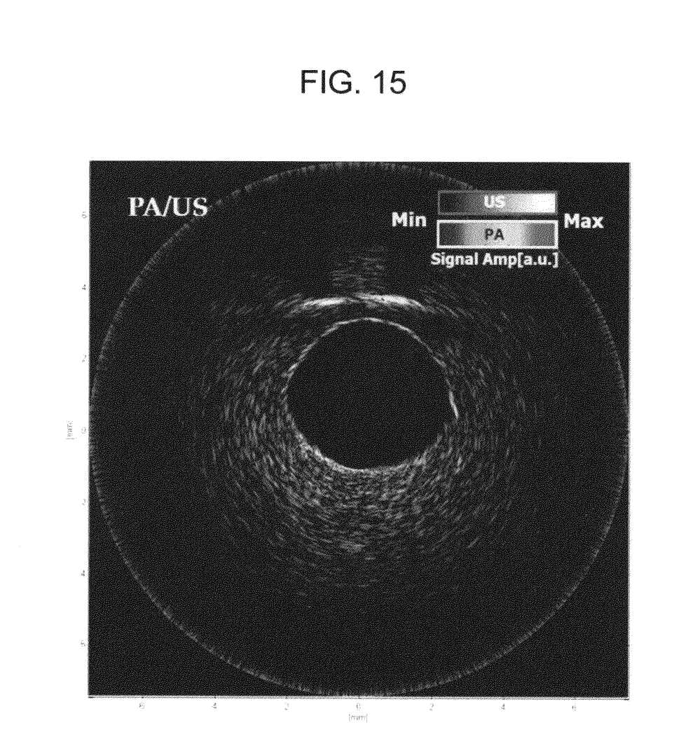

FIG. 14 is a diagram illustrating the ultrasound image (US Image) and the photoacoustic image (PA Image) acquired by rotating the catheter head over the range of 360 degrees in the state of FIG. 13. FIG. 15 is a diagram illustrating an image obtained by superimposing the photoacoustic and ultrasound images of FIG. 14. In FIGS. 14 to 15, a shape of a hole, a white tape and a black tape of the phantom can be identified from the image obtained by superimposing the photoacoustic image and the ultrasound image.

Therefore, in the case where the image acquisition system according to the present invention is applied to a blood vessel to obtain ultrasound/photoacoustic images, a harmful lesion which is full of lipid called vulnerable plaque can be identified. Particularly, by using laser in a wavelength band where the PA signal for the lipid is strongly generated, the lesion which is full of the lipid can be accurately identified. In addition, in the case of outputting a single image obtained by superimposing the ultrasound image and the photoacoustic image, the overall shape can be identified from the ultrasound image, and at the same time, the cross section at a specific position can be identified from the photoacoustic image. Therefore, in the case where the image acquisition system according to the present invention is applied to a blood vessel, the shape of the blood vessel can be accurately identified, and at the same time, a type of the lesion can be determined.

In addition, in the image acquisition system according to the present invention, by rotating the catheter head over the range of 360 degrees, the images of the inner portion of the human body in all directions can be acquired.

An endoscopic catheter for detection of ultrasound and photoacoustic signals according to the present invention and an ultrasound/photoacoustic image acquisition system using the endoscopic catheter can be widely used in a medical image diagnosis field using an endoscope and various applications in industrial fields according to use purposes and methods due to capability of acquisition of high-resolution ultrasound/photoacoustic images.

While the present invention has been particularly shown and described with reference to exemplary embodiments thereof, it will be understood by those skilled in the art that various changes in form and details may be made therein without departing from the spirit and scope of the present invention as defined by the appended claims.

What is claimed is:

1. A catheter for detection of ultrasound and photoacoustic signals, comprising:
a lens optical fiber where a lens is attached to an end of an optical fiber;
an ultrasound transducer which receives and transmits the ultrasound signal;
a cable which is connected to the ultrasound transducer to receive and transmit signals;
a catheter main body where the lens optical fiber and the cable are disposed to pass through an inner portion thereof;
a catheter head which is connected to an end of the catheter main body; and
a membrane which surrounds a surface of the catheter head,
wherein the end of the lens optical fiber where the lens is attached and the ultrasound transducer are fixedly attached to the catheter head, and the ultrasound transducer receives the ultrasound signal and the photoacoustic signal.

2. The catheter according to claim 1, wherein the catheter head includes:
a head main body having a predetermined length;
a hollow portion which is formed in an inner portion of the head main body along a longitudinal direction of the head main body;
an opening portion which is formed to be connected to an end of the hollow portion; and
a groove which is formed on a surface of the head main body along the longitudinal direction of the head main body,
wherein the ultrasound transducer is attached to the hollow portion, the lens optical fiber is attached to the groove, and the ultrasound transducer and the lens of the lens optical fiber are disposed so that front surfaces thereof are directed to the opening portion of the catheter head.

3. The catheter according to claim 1, wherein a space between the membrane and the catheter head is filled with deionized water.

4. The catheter according to claim 1, wherein the membrane is configured with a thin film of a chemical material, and the thin film is configured with a material and a thickness having a low degree of attenuation of the ultrasound signal so as to allow the ultrasound signal to be transmitted and have a transparent property.

5. The catheter according to claim 1, wherein the lens of the lens optical fiber is attached to the catheter head so that the light transmitted through the lens optical fiber emits in the direction where the ultrasound transducer is directed.

6. An ultrasound/photoacoustic image acquisition system comprising:
a light source;
a beam splitter which splits light output from the light source into first light and second light;
an optical fiber which transmits the first light;
a photodetector which detects the second light and converts the detected second light to an electrical signal to transmit the electrical signal as a reference signal;
a catheter where a lens optical fiber and an ultrasound transducer are attached to a catheter head so that the catheter can receive and transmit the ultrasound signal or receive a photoacoustic signal; and
a signal output unit which is connected to the catheter and the photodetector to receive the reference signal from the photodetector or receive the ultrasound signal or the photoacoustic signal from the ultrasound transducer of the catheter to generate an ultrasound image and a photoacoustic image by using the received signals,
wherein the optical fiber is connected to the lens optical fiber of the catheter to transmit the first light to the lens optical fiber.

7. The ultrasound/photoacoustic image acquisition system according to claim 6, wherein the light source is a variable wavelength pulse laser.

8. The ultrasound/photoacoustic image acquisition system according to claim 6, further comprising a rotation stage of which one side is connected to a rotary joint,
wherein a cable connected to the optical fiber and the signal output unit is connected to an input terminal of the rotary joint, and
wherein the rotation stage is connected to an end of the catheter through a jig.

9. The ultrasound/photoacoustic image acquisition system according to claim 6, wherein the catheter includes:
the lens optical fiber which a lens is attached to an end of the optical fiber;
the ultrasound transducer which receives and transmits the ultrasound signal;
a cable which is connected to the ultrasound transducer to receive and transmit signals;
a catheter main body where the lens optical fiber and the cable are disposed to pass through an inner portion thereof;
a catheter head which is connected to an end of the catheter main body; and
a membrane which surrounds a surface of the catheter head,
wherein the end of the lens optical fiber where the lens is attached and the ultrasound transducer are fixedly attached to the catheter head, and the ultrasound transducer receives the ultrasound signal and the photoacoustic signal.

10. The ultrasound/photoacoustic image acquisition system according to claim 9, wherein the catheter head includes:
a head main body having a predetermined length;
a hollow portion which is formed in an inner portion thereof along a longitudinal direction of the head main body;
an opening portion which is formed to be connected to an end of the hollow portion; and
a groove which is formed on a surface thereof along the longitudinal direction of the head main body,
wherein the ultrasound transducer is attached to the hollow portion, the lens optical fiber is attached to the groove, and the ultrasound transducer and the lens of the lens optical fiber are disposed so that front surfaces thereof are directed to the opening portion of the catheter head.

11. The ultrasound/photoacoustic image acquisition system according to claim 10, wherein a space between the membrane and the catheter head is filled with deionized water.

12. The ultrasound/photoacoustic image acquisition system according to claim 9, wherein the signal output unit includes:
a pulser receiver which receives the photoacoustic signal and the ultrasound signal from the catheter; and
an oscilloscope which receives the photoacoustic signal and the ultrasound signal from the pulser receiver and receives the reference signal from the photodetector to generate a photoacoustic image and an ultrasound image by using the received signals.

13. The ultrasound/photoacoustic image acquisition system according to claim 6, wherein the signal output unit outputs the photoacoustic image and the ultrasound image individually or outputs an image obtained by superimposing the photoacoustic image and the ultrasound image.
